(19) United States
(12) Patent Application Publication
(10) Pub. No.: US 2013/0243852 A1
(43) Pub. Date: Sep. 19, 2013

(54) ADJUVANT COMPOSITIONS AND METHODS OF POTENTIATING HDAC INHIBITORS USED TO TREAT VARIOUS DISEASES

(71) Applicant: Robert Sabin, Mill Neck, NY (US)

(72) Inventor: Robert Sabin, Mill Neck, NY (US)

(21) Appl. No.: 13/891,164

(22) Filed: May 9, 2013

Related U.S. Application Data
(63) Continuation-in-part of application No. 13/801,470, filed on Mar. 13, 2013.
Application No. 13/801,470 is a continuation-in-part of application No. 12/152,752, filed on May 16, 2008.
Application No. 12/152,752 is a division of application No. 10/888,576, filed on Jul. 9, 2004, now Pat. No. 7,449,196.
Application No. 13/801,470 is a continuation-in-part of application No. 11/891,613, filed on Aug. 10, 2007, now abandoned.
Application No. 11/891,613 is a continuation of application No. 10/888,576, filed on Jul. 9, 2004, now Pat. No. 7,449,196.
Application No. 13/801,470 is a continuation-in-part of application No. 11/192,752, filed on Jul. 29, 2005.
Application No. 11/192,752 is a continuation-in-part of application No. 10/888,576, filed on Jul. 9, 2004, now Pat. No. 7,449,196.
(60) Provisional application No. 60/666,135, filed on Mar. 29, 2005.
(60) Provisional application No. 60/598,179, filed on Aug. 2, 2004.

(30) Foreign Application Priority Data
Jul. 8, 2005 (US) PCT/US05/24272

(51) Int. Cl. A61K 31/721; A61K 33/26; A61K 31/185; A61K 9/50; A61K 38/28

(52) U.S. Cl. CPC A61K 31/721; A61K 9/50; A61K 38/28; A61K 31/185; A61K 33/26
USPC 424/451; 514/59; 424/490; 514/6.5; 424/648; 424/647

(57) ABSTRACT

A composition and method for potentiating, sensitizing, and/or amplifying at least one HDAC inhibitor targeting at least one disease in a patient is provided. In one embodiment, the composition is administered to potentiate, sensitize and/or amplify an HDAC inhibitor targeting at least one cancer.

### RELATED APPLICATIONS

This application is a continuation-in-part of application Ser. No. 13/801,470, filed Mar. 13, 2013; continuation-in-part of application Ser. No. 12/152,752 filed May 16, 2008; continuation-in-part of application Ser. No. 11/891,613 filed Aug. 10, 2007; continuation-in-part of application Ser. No. 11/192,752 filed Jul. 29, 2005 and Published as U.S. Patent Application Publication No. 2006/0147512 A1 on Jul. 6, 2006; and continuation-in-part of application Ser. No. 10/888,576 filed Jul. 9, 2004, now U.S. Pat. No. 7,449,196 issued Nov. 11, 2008; and claims priority under 35 U.S.C. 120 therefrom. This application is also based in part upon provisional application No. 60/598,179 filed on Aug. 2, 2004 and upon provisional application No. 60/666,135, filed on Mar. 29, 2005, and claims benefit under 35 U.S.C. 119(e) therefrom. This application is also based in part upon PCT/US05/24272 and claims benefit under 35 U.S.C. 119(b) therefrom. The content of each application is expressly incorporated herein by reference.

### TECHNICAL FIELD

The present disclosure relates generally to a Composition and method for potentiating, sensitizing, and/or amplifying at least one HDAC inhibitor targeting at least one disease.

### BACKGROUND OF THE INVENTION

All patents, scientific articles and other documents mentioned herein are expressly incorporated by reference as if reproduced in full.

Chemotherapy (also called Chemo) is a type of cancer treatment that uses drugs to destroy cancer cells. Chemotherapy works by stopping or slowing the growth of cancer cells, which grow and divide quickly. But it can also harm healthy cells that divide quickly, such as those that line your mouth and intestines or cause your hair to grow. Damage to healthy cells may cause side effects. Often, side effects get better or go away after chemotherapy is over. (See: “Chemotherapy and You: Support for people with Cancer”, National Cancer Institute, U.S. Department of Health and Human Services, National Institutes of Health, page 1). More often than not, some of the side effects are permanent; the patient is never the same as before treatment.

Because chemotherapy is non-specific to the cancer cell, there are horrific adverse drug effects or side effects to normal tissues. The National Cancer Institute publishes chemotherapy side effects sheets because of widespread debilitating deadly side effects of non-specific chemotherapy to treat cancer. These side effect sheets are available on the following topics: anemia, appetite changes, bleeding problems, constipation, diarrhea, fatigue, (feeling weak and very tired), hair loss, infection, memory changes, mouth and throat changes, nausea and vomiting, nerve changes, pain, sexual and fertility changes in women, sexual and fertility changes in men, skin and nail changes, swelling, and there are additional adverse drug effects not listed here.

Cancer is commonly viewed as, at best, minimally controlled by modern medicine, especially when compared with other major diseases. Indeed, the age-adjusted mortality rate for cancer is about the same in the 21st century as it was 50 yrs ago, whereas rates for cardiac, cerebrovascular, and infectious diseases have declined by about two thirds. 1. (See Harold Varmus, “The New Era in Cancer Research,” SCIENCE, Vol. 312, 26 May 2006, pg 1162-1165)

Therefore it follows that there is a huge unmet medical need for less or non-toxic cancer therapies, which are safe and effective. To this end, very specific molecular targets unique to cancer cells are being developed, which, for the most part will, it is hoped, spare normal tissues, and be less toxic. See Varmus. “A New Era in Cancer Therapeutics is approaching”. Varmus, Op. Cit

The NCI publishes “Fact Sheet: Targeted Cancer Therapies” National Cancer Institute at the National Institutes of Health, reviewed 5 Dec. 2012, which discloses that targeted cancer therapies are drugs or other substances that block the growth and spread of cancer by interfering with specific molecules involved in tumor growth and progression. Because scientists call these specific molecules molecular targets, therapies that interfere with them are sometimes called molecular targeted drugs or molecular targeted therapies, or other similar names.

Targeted cancer therapies that have been approved for use in specific cancers include: 1) therapies that include drugs that interfere with cell growth signaling or tumor blood vessel development, 2) therapies that promote the specific death of cancer cells, 3) therapies that stimulate the immune system to destroy specific cancer cells, and 4) therapies that deliver toxic drugs to cancer cells.

Other targeted therapies modify the function of proteins that regulate gene expression and cellular functions. Certain HDAC (histone deacetylases) inhibitors are effective against human cancers. For example, vorinostat (also known under the Tradename ZOLINZA®) was approved by the U.S. Food & Drug Administration (FDA) in October 2006 for treatment of cutaneous T-cell lymphoma (CTCL) to treat cutaneous T-cell lymphoma (CTCL) that has persisted, progressed, or recurred during or after treatment with other medicines. This small-molecule drug inhibits the activity of a group of enzymes called histone deacetylases (HDACs) which remove small chemical groups called acetyl groups from many different proteins, including proteins that regulate gene expression. By altering acetylation of these proteins, HDAC inhibitors can induce tumor cell differentiation, cell cycle arrest, and apoptosis. Romidepsin (also known under the Trade name ISTODAX®) is another FDA approved HDAC inhibitor that was approved by the U.S. FDA in November 2009 for treatment of cutaneous T-cell lymphoma (CTCL) cancer.

Furthermore, as noted in Li, Virginia, “Panobinostat Combination May be Effective in Relapsed and Velcade-Refractory Multiple Myeloma (ASH 2011)”, published in myelomabeacon.com, Jan. 18, 2012, the HDAC inhibitor panobinostat may be effective in combination for treating multiple myeloma cancer. As also noted in Hempel, Chris, “Novartis Phase 3 cancer drug Panobinostat may correct Niemann Pick Type C cholesterol defect through inhibiting histone deacetylase (HDAC)”, addiandcassifund.com, 21 Mar. 2011) panobinostat “is effective in cholesterol homeostatis in cultured NPC1 mutant fibroblasts to almost normal levels within 72 hours when used at 40 nM.” Hempel also reports HDAC inhibitors may be effective in treating neurodegenerative diseases, such as Alzheimer's disease, Huntington's disease, amyotrophic lateral sclerosis (ALS) disease, among others.

There are many more HDAC inhibitors undergoing clinical trials. HDAC inhibitors are being investigated in clinical trials as monotherapies or in conjunction with other treatments such as chemotherapy, biologic therapy or radiation therapy. (Citation: Shabason, et al “HDAC inhibitors in cancer care”, Howard Hughes Medical Institute-National Institutes of Health Research Scholar, Radiation Oncology Branch, National Cancer Institute, Bethesda, Md., 24 Feb. 2010) (Citation: Ocio, et al, “In vitro and in vivo rationale for the triple combination of panobinostat (LBH589) and dexamethasone with either bortezomib or lenalidomide in multiple myeloma”, Centro de Investiacion del Cancer, IBMCC/CSIC-Universidad de Salamanca, Spain; Department of Hematology, University Hospital of Salamanca, Salamanca, Pain and Novartis Institutes for Biomedical Research, Cambridge Mass., 13 Oct. 2009) (Citation: LeMoine, etal, “The pan-decetylase inhibitor panobinostat induces cell death and synergizes with everolimus in Hodgkin lymphoma cell lines” Blood, American Society of Hematology, Washington DC republished online, 9 Mar. 2012) (Citation: Budman, etal, “The histone deacetylase inhibitor panobinostat demonstrates marked synergy with conventional chemotherapeutic agents in human ovarian cancer cell lines”, www.ncbi.nlm.nih.gov/pubmed/20533074, 29 (6):1224-9, Dec. 2011)

However, according to Dinarello et al, in Histone Deacetylase Inhibitors for Treating a Spectrum of Diseases Not Related to Cancer, Molecular Medicine, 2011 May-June; 17 (5-6); 333-352, published online 2011 May 5, dol:10.2119/molmed2011.00116, there are many studies for treatment of non-cancerous diseases with HDAC inhibitors, such as inflammatory diseases, auto immune diseases, chronic neuro degenerative diseases, sickle cell disease, diabetes, heart failure, joint cartilage diseases, graft vs host disease in transplant recipients, lupus, rheumatoid arthritis, osteoarthritis, multiple sclerosis, inflammatory bowel disease, atherosclerosis, sepsis, gout, Alzheimer's disease, acute brain trauma, amyotrophic lateral sclerosis, polycythemia and HIV infection.

As noted in M A Glozak and E Seto, of the H. Lee Moffitt Cancer Center & Research Institute, Tampa, Fa., USA,
“Histone deacetylases (HDACs) regulate the expression and activity of numerous proteins involved in both cancer initiation and cancer progression. By removal of acetyl groups from histones, HDACs create a non permissive chromatin conformation that prevents the transcription of genes that encode proteins involved in tumorigenesis. In addition to histones, HDACs bind to and deacetylate a variety of other protein targets including transcription factors and other abundant cellular proteins implicated in control of cell growth, differentiation and apoptosis. This review provides a comprehensive examination of the transcriptional and post-translational mechanisms by which HDACs alter the expression and function of cancer-associated proteins and examines the general impact of HDAC activity in cancer.
Oncogene (2007) 26, 5420-5432; doi:10.1038/sj.onc.1210610”

Glozak goes on to state on page 5428 that “The potential use of HDAC inhibitors in the clinic to treat cancer and other disorders is exciting.”

Without being limited, held or bound to any particular therapy or mechanism of action, it is believed that out of the 18 HDACs in humans, eleven are zinc dependent. Thus it follows that many of the HDAC inhibitors are targeting this zinc dependence. (See Marks, P, “Histone Deacetylases and Histone Deacetylase Inhibitors: Development and Discovery of New Targeted Anticancer Drugs”, Memorial Sloan-Kettering Cancer Center, 2013) (See Codd R, “Zn (II)-dependent histone deacetylase inhibitors: suberoylanilide hydroxamic acid and trichostatin A”, Int J Biochem Cell Biol, 41(4):736-9, Apr. 2009) (See Breslow R, “Zinc”, Chemical & Engineering News, 2003). It is thought that vorinostat binds zinc; thus zinc is a reasonable target. (See NCI Drug Dictionary, www.cancer.gov/drugdictionary?cdrid=37944) (See Wang etal, “Zinc binding in HDAC inhibitors: a DFT study”, J Org Chem, Vol 72(14):5446-9, 6 Jul. 2007) (See Chen etal, “Computational Exploration of Zinc Binding Groups for HDAC Inhibition, Journal of Organic Chemistry, 14 Apr. 2013) Because HDACs so far do not have the activity or robustness as stand-alone drugs generally, various combinations are under investigation in clinical trials. (See Shabason op cit) (See Ocio, op cit) (See Budman, B R, op cit) (See LeMoine, op cit). There is a multitude of combination chemotherapy relating to HDAC inhibitors combined with other cancer drugs because the HDAC inhibitors do not seem to have the strength as stand-alone treatment for many cancers. The problem is that combining HDAC inhibitors with other cytotoxic drugs or other cancer therapeutics or biologic drugs is that there may be an additive effect in activity, but clearly, there is an additive increase in toxicity. It would be very advantageous to have a non-toxic agent that could be combined with the HDAC inhibitors to potentiate and synergize with them so that activity is increased, the quantity of the HDAC inhibitor is reduced and toxicity from the HDAC inhibitor is also reduced.

Combination chemotherapy combines many agents, a cocktail of many anti-cancer drugs at smaller quantities so that the toxicity of each drug is reduced, but it is the same as giving 100% of one of the other drugs. The effect on the tumor is additive. Moreover, the Holy Grail of cancer medicine is a combination which is additive and synergistic so that less of each drug in the combination chemotherapy is used with increased activity and less toxicity then using 100% of a single drug. Moreover, often these combinations have increased toxicity as is well known to those in the art. (See Fox M, “Emil Frei III Who Put Cancer Cures in Reach, Dies at 89”, The New York Times, 4 May 2013) (See Sabin tumor patent U.S. Pat. No. 7,449,196, the contents of which are expressly incorporated herein by reference). As provided in U.S. Pat. No. 7,449,196, in ten tumor cell lines, the addition of iron dextran in vitro at 60 ug/ml increased cytotoxicity, reduced the amount of copper drug required and reversed drug resistance to copper drug in several cell lines and restored high activity. U.S. Pat. No. 5,202,353 of Schroth discloses that iron added to cupric hydroxide increases the antibacterial effect of cupric hydroxide in vitro and reverses copper resistance of bacteria restoring high activity. (See FIGS. 1A and 1B of U.S. Pat. No. 5,202,353) This copper drug of Sabin '196 is exquisitely suited with its dextran encapsulated copper hydroxide to be optionally used along with the HDAC inhibitor and iron dextran to further increase cytotoxity against cancer and precancerous conditions, such as the following precancerous conditions, without being limited, held or bound to, namely, actinic keratosis, Barrett's esophagus, atrophic gastritis, cervical dysplasia, multiple myeloma and myelodisplastic syndrome (MDS). Applicant has administered his copper drug and iron dextran together uneventfully to dozens of laboratory animals. See U.S. Patent application publication 2006/0147512 of Sabin and U.S. Pat. No. 7,449,196 of Sabin. Moreover, Schroth further discloses the addition of added iron increases activity. The more iron added, the more activity. U.S. Patent Application Publication 20060147512, the contents of which are expressly incorporated herein by reference, to Sabin discloses anti-tumor activity in mouse xenographs with lung and breast tumors in mice with intratumoral injection of copper dextran without toxicity and generation of ROS. Sabin further discloses the results of the well-known NCI60 human tumor cell line anti-cancer drug screen. (see Ang Sun, Robert Sabin et al, “Cupric hydroxide-dextran induces cancer cell death both in vitro and in vivo through ROS generation and activation of the intrinsic apoptosis pathway”, undated manuscript). Iron dextran in all cases adds to the cytotoxicity of the copper dextran so that less copper is required to achieve an IC50 in all cases. Moreover, the same result is obtained in the IC90 with less copper being required to achieve an IC90 in all cell lines. Moreover, the addition of iron further increased the IC100 in many cell lines. The addition of iron also reversed resistance to copper in some cell lines to IC50's, IC90's, and IC100's which did not achieve any of the aforementioned IC's without the addition of added iron (see Ang Sun, Robert Sabin et al, “Cupric hydroxide-dextran induces cancer cell death both in vitro and in vivo through ROS generation and activation of the intrinsic apoptosis pathway” op cit).

Clearly there is room for improvement for a scalable, druggable, cheapable (cost effective) and brainable (capable of being administered without straining the mental capacity of the medical team) cancer therapy with acceptable toxicity to treat a broad range of cancers.

Iron and copper can damage DNA (see Sagripanti, DNA Damage Mediated by Metal Ions with Special Reference to Copper and Iron) and inactivate HIV (See Sagripanti, “Cupric and Ferric Ion Inactivate HIV”, AIDS Research and Human Retroviruses, Vol. 12, No. 4, 1996, pgs. 333-336).

Iron dextran has been administered since 1955 intramuscularly and IV since 1971. There have been hundreds of millions of administrations in the United States and throughout the world. All intravenous (IV) iron agents are colloids that consist of spheroidal iron-carbohydrate nanoparticles. At the core of each particle is an iron-oxyhydroxide gel. The core is surrounded by a shell of carbohydrate that stabilizes the iron-oxyhydroxide, slows the release of bioactive iron, and maintains the resulting particles in colloidal suspension. (see Danielson, “Structure, Chemistry, and Pharmacokinetics of Intravenous Iron Agent”, J Am Soc Nephrol 15: 93-98, 2004)

Moreover, after IV administration, iron dextran mixes with plasma and then enters the RES system directly from the intravascular fluid department. Iron dextran, in the plasma, crosses the BB barrier, the bone marrow, the lymphatic system and anywhere plasma traffics, iron dextran traffics, which is everywhere. (see Danielson) Iron dextran also is taken up by red blood cells (in vitro activity against malaria with copper drug) which means dextran is taken up by RBC.

In contrast to other parenteral iron preparations, because of the core/shell configuration of iron dextran, it has been given intravenously in doses as high as 2-3 gm without apparent toxicity from the release of excessive free iron into the circulation. The reticuloendothelial system plays a key role in the utilization of the remaining portion of the iron dextran complex. After an intravenous administration of large doses, 2000-3000 mg of the iron dextran complex, complete removal of the material from plasma takes as long as 2-3 weeks. With infusions of up to 500 mg of the material at one time, clearance into the reticuloendothelial system is exponential. When doses in excess of 500 mg are administered, the initial clearance rate does not exceed 10-20 mg/hr., the maximum removal rate of the reticuloendothelial system. Once cleared, the material is readily visible as iron stores on Prussian blue stain of marrow stroma. (see Henderson & Hillman, “Characteristics of Iron Dextran Utilization in Man”, BLOOD, Vol. 34, No 3 (September) 1969, pgs. 357-370) (see Marchasin & Wallerstein, “The Treatment of Iron-Deficiency Anemia with Intravenous Iron Dextran”, BLOOD, Vol. 23, No. 3 (March) 1961, FIG. 1) Note: lack of toxicity. Because iron dextran is a core/shell formulation, with the dextran chains encapsulating the beta ferric oxyhydroxide core, the cellular toxicity of iron is severely curtailed so that doses many times that of iron salts may be administered safely. A fraction of a common three-gram dose of iron dextran IV using iron salts is fatal. There are thousands of papers published about iron dextran. It has been used extensively with kidney dialysis and cancer and more. Published experience with more than 1000 patients in clinical trials in oncology alone involving the use of IV Fe suggests minimal toxicity and substantial benefits are experienced when high molecular weight iron dextran is avoided. (see Auerbach, “Intravenous Iron Optimizes the Response to Recombinant Human Erythopoietin in Cancer Patients with Chemotherapy-Related Anemia: A Multicenter, Open-Label, Randomized Trial”, Journal of Clinical Oncology, Vol. 22, No. 7, Apr. 1, 2004, pgs. 1301-7) Moreover, 41 cancer patients were administered total dose infusion of iron dextran from 1000 mg/one gram to 3000 mg/three grams with little toxicity. (See Auerbach, JCO)

Iron dextran is also used for livestock throughout the world and is FDA approved for this purpose. Iron dextran is generic and with the human FDA approved version INFED® costing about 100 times more than the veterinary version. The previous PK studies from Henderson & Hillman and Marchasin used IMFERON®, which is no longer available. The current FDA approved iron dextran INFED® is far more robust with a longer plasma half-life than IMFERON®. (see Table 3, U.S. Pat. No. 5,624,668 of Lawrence etal.)

As provided in U.S. Patent Application Publication 20060147512 of Sabin, applicant has administered iron dextran to Cyno Mulgus monkeys at 400 and 500 mg/kg of body weight without toxicity. There is a striking, impressive therapeutic index with iron dextran so that cancer patients will likely receive no more than 50 mg/kg of body weight, 10 percent of the dose safely administered by the total dose infusion method to Cyno Mulgus monkeys with little toxicity.

### SUMMARY OF THE INVENTION

This disclosure relates to a method for potentiating, sensitizing, and/or amplifying at least one HDAC inhibitor targeting at least one disease including the steps of:

forming a composition including a colloidal solution having a core of at least a biologically acceptable insoluble iron compound, insoluble or highly insoluble in water or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier and

administering the composition to the patient to potentiate, sensitize, and/or amplify the at least one HDAC inhibitor targeting the at least one disease in a patient.

Preferably the iron compound is iron dextran. The sheath, shell, polymeric shell, cover, casing, encoating, jacket or combination thereof prevents immediate chemical interaction of the core with the surrounding environment. This is known in the art as a core/shell configuration. In one embodiment, the Composition is administered to potentiate, sensitize and/or amplify at least one HDAC inhibitor targeting at least one cancer. The composition is an adjuvant pharmaceutical for use with HDAC inhibitors. Adjuvants pharmaceuticals are agents that aid or increase the action of the principal drug or that affect the absorption, mechanism of action, metabolism, or excretion of the primary drug in such a way as to enhance its effects.

This disclosure also relates to a Composition for potentiating, sensitizing, and/or amplifying at least one HDAC inhibitor targeting at least one disease comprising a colloidal solution having a core of at least a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier. The sheath, shell, polymeric shell, cover, casing, encoating, jacket or combination thereof prevents immediate chemical interaction of the core with the surrounding environment. This is known as a core/shell configuration in the art and is also disclosed in U.S. Pat. No. 7,449,196 of Sabin, the contents of which are expressly incorporated in its entirety herein by reference. In one embodiment, the composition is administered to patients taking at least one HDAC inhibitor with or without other conventional treatments. In one embodiment, the sheath, shell, polymeric shell, cover, casing, encoating, or jacket is dextran, a polyglucose, polysaccharide with its extensive history of clinical use in millions of patients.

The patient is monitored regularly to determine the level and/or presence of the disease. The composition may be re-administered at intervals determined to be medically necessary by the physician, based on the results of the monitoring.

The present invention is advantageously a safe and effective Composition which employs bio-compatible materials which are native to the body (iron and glucose) and feed every cell in the body to selectively amplify and potentiate HDAC inhibitors so that targeted cells are killed or become cytostatic with acceptable toxicity .

### DETAILED DESCRIPTION

Without limitation, these and other objects, features, and advantages of the present invention, will become apparent to those with skill in the art after review of the following detailed description of the disclosed embodiments. While not being limited, held or bound to any particular theory or mechanism of action, the applicant discloses the detailed description of the invention.

This disclosure is directed to a method for potentiating, sensitizing, and/or amplifying at least one HDAC inhibitor targeting at least one disease in a patient comprising:

forming a composition including a colloidal solution having a core of at least a biologically acceptable water insoluble iron or highly water insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier; said sheath, shell, polymeric shell, cover, casing, encoating, jacket or combination thereof preventing immediate chemical interaction of the core with the surrounding enviromnent; and

administering the composition to the patient to potentiate, sensitize, and/or amplify at least one HDAC inhibitor targeting the at least one disease in the patient.

This is known as a core/shell configuration in the art and is also disclosed in U.S. Pat. No. 7,449,196 of Sabin, the contents of which in its entirety are expressly incorporated herein by reference. While the present invention is directed broadly to a method for treating any diseases capable of being effectively treated with HDAC inhibitors, one embodiment of the invention is directed to administering the composition to potentiate at least one HDAC inhibitor targeting at least one cancer. In other embodiments, the composition is administered to potentiate at least one HDAC inhibitor targeting a disease such as pre-cancer, multiple myeloma, and/or any disease where the diseased cell uses a zinc dependent HDAC (also referred to herein as a zinc targeted HDAC inhibitor). In yet another embodiment, the composition is administered to potentiate at least one HDAC inhibitor targeting a disease such as actinic keratosis, Barrett's esophagus, atrophic gastritis, cervical dysplasia, and myelodysplastic syndromes (MDS). In another embodiment, the disease is selected from the group consisting of inflammatory diseases, auto immune diseases, chronic neuro degenerative diseases, sickle cell disease, diabetes, heart failure, joint cartilage diseases, graft vs host disease in transplant recipients, lupus, rheumatoid arthritis, osteoarthritis, multiple sclerosis, inflammatory bowel disease, atherosclerosis, sepsis, gout, Alzheimer's disease, acute brain trauma, amyotrophic lateral sclerosis, polycythemia and HIV infection. The Composition is an adjuvants pharmaceutical that aids or increases the action of the principal drug or that affect the absorption, mechanism of action, metabolism, or excretion of the primary drug in such a way as to enhance its effects.

The Composition is administered to patients taking at least one HDAC inhibitor with or without conventional treatments which attack the diseased cells. In one embodiment, the composition is administered to patients taking at least one HDAC inhibitor anti-tumor agent with or without conventional cancer treatments which attack the cancer cells. In one embodiment, HDAC inhibitor anti-tumor agents are selected from the group consisting of panobinostat, givinostat, belinostat, vorinostat, valproic acid, romidepsin, disulfram, certain interleukins, and combinations thereof. The patient is monitored regularly to determine the level and/or presence of the disease. The composition may be re-administered at intervals determined to be medically necessary by the physician, based on the results of the monitoring.

The presently disclosed Composition is an adjuvant for HDAC inhibitors in all disease indications where they are currently indicated and will be indicated in the future. The Composition used as an adjuvant simply makes the HDAC inhibitors work better. There is thought to be no anti-tumor activity with iron dextran as a stand-alone treatment.

While not being limited, held or bound to any particular theory or mechanism of action, with regard to cancer treatment it is thought that vorinostat and romidepsin block intracellular zinc in specific places in the cancer cell, so that the presently disclosed composition is believed to attack zinc by antagonism to zinc, displace zinc to the zinc containing catalytic domain of the HDACs, and replace zinc to the zinc containing catalytic domain of the HDACs, with iron respectively. The composition is believed to complement the activity of zinc targeted HDAC inhibitors, of which there are thought to be 11 out of 18 human HDAC's containing zinc.

The combination of the present Composition (e.g. iron dextran) with HDAC inhibitors increases activity and reduces toxicity. Since vorinostat and other Class 1 and Class 2 HDAC inhibitors (Citation Histone deacetylase inhibitor from Wikipedia, http://en.wikipedia.org/wiki/Histone_deacetylase_inhibitor) target zinc binding to the zinc-containing catalytic domain of the HDACs, the present composition also targets zinc by utilizing what is thought to be high levels of iron intracellularly as a zinc antagonist. Iron replaces other vital minerals such as zinc, copper, manganese, and many others in hundreds or even thousands of enzyme binding sites. This causes the enzymes to malfunction. (see Wilson L, “Chronic Acquired Iron Overload—A Disease of Civilization”, http://drlwilson.com/articles/IRON.htm, August 2011) Zinc and iron are well known in the art to be antagonistic towards one another. (See Kordas and Stoltzfus, “New Evidence of Iron and Zinc Interply at the Enterocyte and Neural Tissues,” Journal of Nutrition, Vol 134, No. 6, 1295-1298, 1 Jun. 2004)

Since zinc is a target of the HDAC inhibitors (see NCI Drug Dictionary—vorinostat op cit), the composition seeks to target zinc through a different method. In one embodiment, the present treatment method includes loading cancer cells up with iron which is thought to be a zinc antagonist to potentiate the activity of the HDAC inhibitors, such as vorinostat, which target zinc binding. (See JOC, Wang,op cit)

The composition is comprised of, at least, nanoparticles with an insoluble iron compound core. The nanoparticles of an insoluble iron compound core encapsulated with dextran are also referred to herein as “iron dextran”. This is known as a core/shell configuration. It is understood that the use of “dextran” in “iron dextran” includes other encapsulating material as further disclosed herein and is not strictly limited to dextran per se. The term “insoluble” with respect to iron compounds refers herein to a compound which is insoluble or highly insoluble in water. These cores may be encapsulated, coated, adsorbed, complexed, or the like, with a protective sheath or jacket which also functions to target cancer. This sheath or jacket may be any combination of materials, such as a glucose or liposome. In another embodiment, the core may be encapsulated with dextran alone or any glucose or combination of sugar-based substances.

In a further embodiment, insoluble iron, may be used as a core to provide synergistic effects of the combination. Any biocompatible iron compound may be used, including without limitation, for example, Fe³⁺, and its salts, iron hydroxide, iron oxyhydroxide, iron oxide, and the like, to iron load the intracellular biological environment, including iron-saturated human holotransferrin.

The nanoparticles of the disclosed Composition preferably must be encapsulated, to avoid toxicity, and surrounded, complexed, or adsorbed by, and bound to, at least one sheath or coat that is preferably composed of a sugar substance, such as a glucose, a saccharide, a polysaccharide e.g. starch, cellulose, dextrans, alginides, chitosan, pectin, hyaluronic acid, pullulan (a bacterial polysaccharide), dextran, carboxyalkyl dextran, carboxyalkyl cellulose and the like. These dextrans can include, for example, those disclosed by Mehvar, supra (2000); and Recent Trends in the Use of Polysaccharides for Improve Delivery of Therapeutic Agents: Pharmacokinetic and Pharmacodynamic Perspectives, Curr. Pharm. Biotech. 4:283-302 (2003), and liposomes coated with dextran as disclosed by Moghimi, et al., Long-Circulating and Target-Specific Nanoparticles: Theory to Practice, Pharm. Rev., 53(2):283-318 (2001) both of which are incorporated herein in their entirety. The sheath encoats, or encapsulates, the disclosed Composition's core and prevents chemical interaction of the core with the surrounding environment, blocking the degradation of the core and the emanation of the iron from the iron compound from the core. The thickness of the sheath may be varied, if desired, by those skilled in the art. Because the sheath is composed primarily of a substance that is not necessarily recognized by the body as foreign matter, the body is less likely to develop a resistance to the Composition. In one embodiment, the sheath can be composed of dextran, also known as macrose, a high molecular weight polysaccharide. Dextran is an ideal candidate for use as a sheath because it is often administered to mammals as a blood plasma substitute or expander, is generally not rejected by the mammalian system, and can remain in the plasma for an extended period of time. Other biocompatible materials for the formation of a polymeric shell, sheath, or jacket can include proteins, polypeptides, oligopeptides, polynucleotides, polysacchrides, lipids and so on. Additional sheath materials include, for example, those of U.S. Pat. Nos. 6,096,331; and 6,506,405, incorporated herein in their entirety. Alternatively, combinations of two or more of the above named materials may be used to form the sheath.

The nanoparticle size of the entire disclosed Composition may be approximately 1 nm to approximately 10,000 nm. In a more preferred embodiment, the particle size may be approximately 15 nm to approximately 500 nm. A most preferred embodiment for particle size is approximately 20 nm to approximately 200 nm. An additional post preferred embodiment is approximately 90 to 100 nm, the exact size of the FDA approved InFed iron dextran tested by laser light scattering.

Without being limited, held, or bound to any particular theory or mechanism of action, it is believed that the Composition, i.e., iron dextran enters the vascular system/blood, traffics throughout the body, crosses the blood brain barrier, enters the marrow, enters the lymphatic system and traffics wherever plasma traffics as an inert entity, and is removed from the plasma by the phagocytic system. The Composition can remain in the mammal's plasma compartment for a period of many days, at least two weeks, depending on the dosage levels. (It is known that iron-dextran can remain in the plasma for weeks, especially when doses are administered above the clearance rate of the mononuclear phagocyte system. The processing of the iron dextran by the phagocytic system is rate limited to a daily maximum amount, leaving the balance for future use.) The sheath may not be immediately recognized as foreign matter by the phagocytic system because it is a sugar-based substance and is not rejected by the mammalian system, allowing the Composition to remain in circulation of the mammal for a longer period than most therapeutics, making it more likely to come into contact with target cells and providing more efficacy with fewer doses than traditional agents. The Composition circulates, via any biological pathway, throughout the body and may contact any cell type. For the most part, the phagocytic system takes up the Composition. Normal, healthy cells generally have very little interaction with the Composition. The Composition that is taken up by the phagocytic system is processed, to a large degree, through the liver in hepatocytes that store glucose and iron and are later released through their appropriate protein carriers to feed and nurture cells of the body. Since sugars and iron are bodily requirements, well known to the phagocytic system, the phagocytic system is able to process, transport, store, or eliminate them with little toxicity, while the Composition potentiates, sensitizes, and/or amplifies at least one adjuvant pharmaceutical or mono pharmaceutical agent targeting at least one cancer in a patient. The Composition, because it is an essential requirement for biological systems, iron and glucose simultaneously feeds and nourishes cells in the body. Far higher dosages of iron dextran may be employed, as opposed to elemental iron salts, for a greater iron loading of cancer cells, and a protracted residence plasma time. In one embodiment, the iron dextran can be administered above the clearance level of the phagocyte system to allow the Composition to remain in the plasma for an extended period of time. (See, Henderson & Hillman, Characteristics of Iron Dextran Utilization in Man, Blood, 34(3):357-375(1969)). (See Marchasin and Wallerstein, The Treatment of Iron-Deficiency Anemia with Intravenous Iron Dextran, BLOOD, The Journal of Hematology, Vol XXIII, No. 3, March 1964) Generally, smaller doses of iron dextran (50-500 mg) are cleared within approximately 3 days, larger doses of iron dextran (>500 mg), however, are cleared at a constant rate of 10-20 mg/hr and are typically associated with increased plasma concentration of iron dextran for as long as 3 weeks.

Since the disclosed composition, iron dextran, is formed of biocompatible materials, it may be administered over an extended period of time as compared to other chemotherapeutic agents. The effective dose or effective amount can vary subject to the evaluation of those of skill in the art in relation to the particular type of disease to be treated, the regimen of administration, the body weight of the subject, the aggressiveness of the disease and the degree in which the subject has been negatively affected by prior therapy.

The disclosed Composition may be administered to a patient in a variety of ways, such as injection, intravenous, subcutaneous, intraperitoneal, intrathecal, intramuscular, intracranial, inhalational, topical, transdermal, or an implantable polymer iron dextran saturated depot or wafer, such as, for example, a Giladel wafer®.

Actual methods for preparing parenterally administerable compounds and adjustments necessary for administration to patients, typically mammals, will be known or apparent to those skilled in the art and are described in more detail in, for example, Remington's Pharmaceutical Science: The Science and Practice of Pharmacy, 20.sup.th Ed., Lippincott, Williams & Wilkins; (2000), which is incorporated herein by reference.

In one embodiment, the colloidal solution includes a core of a biologically acceptable insoluble iron compound encapsulated, encoated, adsorbed, complexed or bound to dextran. A biologically acceptable iron compound as defined herein is an iron compound, which may be used with and within a biological system with little or no detrimental effect, i.e. it does not appreciably alter or appreciably affect in any adverse way, the biological system into which it is introduced. In one embodiment, the insoluble iron compound can be selected from the group consisting essentially of iron oxide, iron hydroxide, and iron oxyhydroxide. Iron dextran is the perfect adjuvant cancer pharmaceutical agent. It traffics all throughout the body including the marrow, the lymphatic system, the interstitial space, the plasma (where it is inert), with the cellular toxicity of iron blocked by the dextran chains surrounding and encapsulating the core. It has a plasma half-life, or residence time inertly in the plasma for say less than one day to say about three weeks or more, from one single I.V. administration, and will be where it is needed in the tumor cell for as long as the HDAC inhibitors are typically administered. Vorinostat is approved for 14 successive days of administration, and will persist in the body after the last 14th day treatment, and the medical team can titrate, or dial in exactly how long they want the adjuvant in the plasma . . . it is also cost-effective and generic . . . and crosses the blood/brain barrier (see Neuwelt, E. A., “Imaging of iron oxide nanoparticles by MR and light microscopy in patients with malignant brain tumours’, Neuropathal Appl Neurobiol. 2004 October; 30(5); 456-71, which discloses that dextran encapsulated/coated nanoparticles cross the blood/brain barrier where brain cancer cells are resident).

First, dextran is a polyglucose. It is well known and established to the scientific and medical community as a plasma volume expander and as the polymeric shell of iron dextran with several hundreds of millions safe administrations. Moreover, according to the FDA approved package insert for INFED®, dextran is metabolized or excreted, iron is stored, and the PK is known. There have been at least 100 million administrations of iron dextran throughout the world for about 59 years.

Dextran is also cheap, generic and best of all, a polyglucose, with cancer cells being phagocytic, with a predilection for glucose. One of the most critical and important parts of this invention is that iron dextran can iron load the cancer cells which are phagocytic with a predilection for glucose with iron without affecting normal cells and causing much toxicity. At this point with the iron dextran potentially loading the cancer cells for weeks, when the HDAC inhibitor is administrated, such as vorinostat, there may be ROS generation and since iron is thought to be a zinc antagonist, it may complement the HDAC inhibitor such as vorinostat which targets zinc to cause cell death or have a cytostatic effect. The vorinostat or other HDAC inhibitors spare normal cells with acceptable toxicity. It is thought that cancer cells are more fragile than normal cells and that the combination of iron loading and vorinostat or other HDAC inhibitors targeting zinc will push the cancer cell over the edge and kill it.

In a preferred embodiment, the insoluble iron core may include iron hydroxide, iron-oxyhydroxide, or iron oxide or other insoluble iron compounds with a shell of dextran or shells as disclosed in U.S. Pat. No. 6,096 331 of Desai (page 9, paragraphs 7-9) and (page 10, paragraphs 1-2). This insoluble iron core is a biological, compatible material that does not significantly change or affect in any detrimental manner, the living system into which it is utilized. U.S. Pat. No. 5,624,668 of Lawrence, for Iron Dextran Formulations, discloses how to manufacture such iron dextran formulation. Lawrence also discloses information about iron dextran formulations in his following paper: “Development and Comparison of Iron Dextran Products, Journal of Pharmaceutical Science and Technology, Vol. 52, No. 5, September-October 1998, pgs 190-197.

Iron dextran has perfect pharmacokinetics as an adjuvant for cancer treatment and is thought to be, through iron loading of cancer cells and thus antagonizing zinc, taken together with the HDAC inhibitors, such as vorinostat which targets zinc with the further addition of ROS generated by iron which damage DNA to effect cell kill. In U.S. Pat. No. 7,449,196 of Sabin the addition of iron dextran to the applicant's copper drug increased ROS production and cytotoxicity in ten major tumor cell lines so that less copper was needed for an IC50 (see Table 1 in Sabin) and also achieve an IC100.

The finesse object of the invention is to iron load (loading dose is defined as a large initial dose of a substance or series of such doses given to rapidly achieve a therapeutic concentration in the body) the cancer cell. It is an object of the present invention to load cancer cells throughout the body with iron dextran which will act as an adjuvant to the HDAC inhibitors, such as vorinostat, which are adjuvants or stand alone pharmaceutical agents to cancer treatments. Those of skill in the art can easily calculate patient weight, blood volume and the amount of iron dextran to be administered to achieve the levels desired and the duration of elevated iron in the plasma. The oncologist, by administering less than one gram to about three grams, can match by one single total dose infusion the duration of treatment of the HDAC inhibitors, say vorinostat, which has an FDA-approved dose of 400 mg/day orally for 14 successive days. The oncologist can titrate in iron dextran to be in the plasma and throughout the body for five, ten, fifteen days, more or less, whichever number of days matches or complements the HDAC inhibitors being used. Most of the world is iron deficient and iron is stored in the body in the liver, generally, about 4-5 grams.

Applicant's compounds of iron dextran are to be administrated by injection, IV IP, etc. (See Administration of Intravenous Iron Dextran, sickle.bwh.harvard.edu)—The medical team can calculate exactly how long the compounds will remain in the plasma, traffic all throughout the body in the plasma, expose most or all of the cancer sites, including but not limited to the lymphatic system, marrow, CNS system, all major organ systems, and cross the blood/brain barrier. By example, U.S. Pat. No. 5,624,668 of Lawrence discloses in Example 3, Table 3, that the sole current FDA approved iron dextran, INFED®, by way of a single 100 mg intravenous dose has a plasma residence time, half life of 34.2 hours so that iron dextran compounds will hit essentially all tumors. This half life is many times extended by doses of 500 mg, 1000 mg, 2000 mg, 3000 mg which have been safely administered to millions of people and hundreds of millions of livestock throughout the world. The medical team treating cancer will decide exactly how long they want the agents to hit the cancer and sanctuary sites. It could be two weeks+ by one single TDI administration. Cancer is well known in the art to be very persistent, stubborn, sneaky, so that applicant's composition will be there 24/7 for weeks; cancer cells have an avidity for glucose, glucose promotes and drives cancer cells, cancer cells take up glucose at higher rates than normal tissue, dextran is a polyglucose, very desirable to cancer cells, See Ekat Kritikou, “Metabolism:Warburg effect revisited”, Nature Reviews Cancer, Vol 8(247), April 2008) the iron dextran being metabolized or excreted and the iron being stored in the liver. In one embodiment, in order to promote the ingestion of the applicant's compounds with a polyglucose sheath, insulin may also be utilized as it promotes the uptake of glucose into cells. (See “Insulin Potentiating Therapy” www.mskcc.org/cancer-care/herb/insulin-potentiation-therapy)

For Example, iron dextran may be administered on Day One, about 24 hours later, or less, the anti-tumor agents/HDAC inhibitors, such as panobinostat, givinostat, belinostat, vorinostat, valproic acid, or others may be administered alone or in combination.

In one embodiment, the FDA-approved iron dextran, INFED®, with a size of about 90 nm (tested with Laser Light Scattering) may be administered at one to three grams to 41 patients with cancer of both genders currently under FDA-approved standard care chemotherapy, radiation or surgery taking or having taken a cancer fighting drug. The INFED iron dextran is administered by the total dose infusion method at from one to three grams. The patients are matched as closely as possible to the 41 patients administered by the total dose infusion method at from one to three grams disclosed in Auerbach's report published in the Journal of Clinical Oncology (Opus Cited). There are little or no adverse drug effects. Sometime later, the most preferred time being 24 hours later, the anti-tumor agent, HDAC inhibitor vorinostat, is administered at the FDA-approved dosage of 400 mg/day orally for 14 successive days exactly as it has previously been administered to thousands of patients. The iron dextran will remain in the plasma and traffic throughout the body which includes crossing the blood/brain barrier, the marrow, the lymphatic system for up to 14 or more days, which is as long as the vorinostat is administered. The medical team can either lengthen or shorten the number of days vorinostat will be administered. Other pharmaceutical cancer drugs may also be administered along with the iron dextran and the HDAC inhibitor vorinostat. This is known in the art as combination therapy. The medical team will monitor the patient and make whatever adjustments may be deemed appropriate for dosages and duration of treatment. The medical team will also repeat administration of both agents as necessary or after an appropriate interval.

In another embodiment, the FDA-approved iron dextran, INFED®, with a size of about 90 nm (tested with Laser Light Scattering) may be administered at the identical dose and route of administration used by Auerbach above, at 50 mg/kg of body weight of iron derived from iron dextran by the total dose infusion method to a 60 kg patient with a cutaneous T-cell lymphoma. Sometime later, 24 hours being the ideal, Romidepsin is administered by IV on Days One, Eight and Fifteen of a 28 day cycle. A preferred dose and route is identical to that administered as previously used by hundreds of patients. Applicant's iron dextran should be in the plasma for at least 21 days by one single total dose infusion administration. Repeat cycles every 28 days provided that the patient continues to benefit from and tolerate the drug or as the medical team measures, evaluates and deems appropriate.

It is understood that any recitation of the Composition used to potentiate, sensitize and/or amplify at least one HDAC inhibitor for the treatment of cancer also expressly includes the treatment of other diseases that are treated with HDAC inhibitors

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention.

It is further known that other modifications may be made to the present invention, without departing the scope of the invention, as noted in the appended Claims.

I claim:

1. A method for potentiating, sensitizing, and/or amplifying at least one HDAC inhibitor targeting at least one disease-in a patient comprising:
forming a composition including a colloidal solution having a core of at least a biologically acceptable insoluble iron compound or mixtures thereof, wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier;
said sheath, shell, polymeric shell, cover, casing, encoating, jacket or combination thereof preventing immediate chemical interaction of said core with the surrounding environment; and
administering the composition to the patient to potentiate, sensitize, and/or amplify the at least one HDAC inhibitor targeting the at least one disease in the patient.

2. The method of claim 1, wherein the at least one HDAC inhibitor is a zinc targeted HDAC inhibitor.

3. The method of claim 1, wherein the at least one HDAC inhibitor is an anti-tumor agent.

4. The method of claim 3, wherein said anti-tumor agent is selected from the group consisting of panobinostat, givinostat, belinostat, vorinostat, valproic acid, disulfram, romidepsin, and combinations thereof.

5. The method of claim 1, wherein the composition includes a mixture of separately formed cores of biologically acceptable insoluble iron compound.

6. The method of claim 1, wherein said step of administering said composition includes separately administering said colloidal solution having a core of at least a biologically acceptable insoluble iron compound.

7. The method of claim 1, further comprising parenterally administering the composition to the patient.

8. The method of claim 1, further comprising orally administering the composition to the patient.

9. The method of claim 1, further comprising transdermally administering the composition to the patient.

10. The method of claim 1 wherein said at least one disease is a cancer disease or a pre-cancer disease.

11. The method of claim 1 wherein said at least one disease is selected from the group consisting of inflammatory diseases, auto immune diseases, chronic neuro degenerative diseases, sickle cell disease, diabetes, heart failure, joint cartilage diseases, graft vs host disease in transplant recipients, lupus, rheumatoid arthritis, osteoarthritis, multiple sclerosis, inflammatory bowel disease, atherosclerosis, sepsis, gout, Alzheimer's disease, acute brain trauma, amyotrophic lateral sclerosis, polycythemia, actinic keratosis, Barrett's esophagus, atrophic gastritis, cervical dysplasia, multiple myeloma, myelodisplastic syndrome (MDS) and HIV infection.

12. The method of claim 1, wherein the composition is administered with or without insulin.

13. The method of claim 1 wherein said sheath, shell, polymeric shell, cover, casing, encoating, jacket or combination thereof includes a substance selected from the group consisting of glucose, a saccharide, a polysaccharide, a carbohydrate, a protein, a dextran, a fat, a liposome, derivatives thereof or combinations thereof.

14. The method of claim 1 wherein said core comprises nanoparticles covered by sheath material.

15. The method of claim 1 wherein said biocompatible iron compound is selected from the group consisting of iron hydroxide, iron oxyhydroxide and iron oxide.

16. A chemical formulation for potentiating, sensitizing, and/or amplifying at least one HDAC inhibitor used in conjunction with the treatment of at least one disease;
a composition including a colloidal solution having a core of at least a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier; said sheath, shell, polymeric shell, cover, casing, encoating, jacket or combination thereof preventing immediate chemical interaction of the core with the surrounding environment; and
at least one HDAC inhibitor.

17. The chemical formulation of claim 16, wherein the at least one HDAC inhibitor is a zinc targeted HDAC inhibitor.

18. The chemical formulation of claim 16, wherein said at least one HDAC inhibitor is selected from the group consisting of panobinostat, givinostat, belinostat, vorinostat, valproic acid, disulfram, romidepsin, and combinations thereof.

19. The chemical formulation of claim 16, wherein the sheath, shell, polymeric shell, cover, casing, encoating, jacket or combination thereof includes a substance selected from the group consisting of glucose, a saccharide, a polysaccharide, a carbohydrate, a protein, a dextran, a fat, a liposome, derivatives thereof or combinations thereof.

20. The chemical formulation of claim 16 in which the core comprises nanoparticles covered by sheath material.

21. The chemical formulation of claim 16 in wherein said biocompatible iron compound is selected from the group consisting of iron hydroxide, iron oxyhydroxide and iron oxide

22. A method for potentiating, sensitizing, and/or amplifying at least one HDAC inhibitor to make the cells of a patient undergoing treatment of a disease more sensitive to the cytopathic, cytostatic activity of the at least one HDAC inhibitor comprising:
administering to said patent undergoing cytotoxic or cytostatic cancer treatment a composition comprising a colloidal solution having a core of at least a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier;
said sheath, shell, polymeric shell, cover, casing, encoating, jacket or combination thereof preventing immediate chemical interaction of the core with the surrounding environment;
administering at least one HDAC inhibitor to said patient; and
administering at least one pharmaceutical for treating said disease.

23. The method of claim 22, wherein the at least one HDAC inhibitor is a zinc targeted HDAC inhibitor.

24. The method of claim 22, wherein said at least one HDAC inhibitor and said at least one pharmaceutical is an anti-tumor agent.

25. The method of claim 22, wherein said at least one HDAC inhibitor is selected from the group consisting of panobinostat, givinostat, belinostat, vorinostat, valproic acid, disulfram, romidepsin, and combinations thereof.

26. The method of claim 22, further comprising parenterally administering the composition to the patient.

27. The method of claim 22, further comprising orally administering the composition to the patient.

28. The method of claim 22, further comprising transdermally administering the composition to the patient.

29. The method of claim 22 wherein said disease is a cancer disease or a pre-cancer disease.

30. The method of claim 22 wherein said disease is selected from the group consisting of inflammatory diseases, auto immune diseases, chronic neuro degenerative diseases, sickle cell disease, diabetes, heart failure, joint cartilage diseases, graft vs host disease in transplant recipients, lupus, rheumatoid arthritis, osteoarthritis, multiple sclerosis, inflammatory bowel disease, atherosclerosis, sepsis, gout, Alzheimer's disease, acute brain trauma, amyotrophic lateral sclerosis, polycythemia, actinic keratosis, Barrett's esophagus, atrophic gastritis, cervical dysplasia, multiple myeloma, myelodisplastic syndrome (MDS), and HIV infection.

31. The method of claim 22, wherein the composition is administered with or without insulin.

32. The method of claim 22 wherein said sheath, shell, polymeric shell, cover, casing, encoating, jacket or combination thereof includes a substance selected from the group consisting of glucose, a saccharide, a polysaccharide, a carbohydrate, a protein, a dextran, a fat, a liposome, derivatives thereof or combinations thereof.

33. The method of claim 22 wherein said core comprises nanoparticles covered by sheath material.

34. The method of claim 22 wherein said biocompatible iron compound is selected from the group consisting of iron hydroxide, iron oxyhydroxide and iron oxide.
